(19) United States
(12) Patent Application Publication
(10) Pub. No.: US 2016/0296538 A1
(43) Pub. Date: Oct. 13, 2016

(54) PREDICTIVE AND RESPONSE BIOMARKER FOR TH-302 ANTI-CANCER THERAPY

(71) Applicant: THRESHOLD PHARMACEUTICALS, INC., South San Francisco, CA (US)

(72) Inventors: Charles Hart, South San Francisco, CA (US); Harold E. Selick, South San Francisco, CA (US); Jessica Sun, South San Francisco, CA (US)

(73) Assignee: THRESHOLD PHARMACEUTICALS, INC., South San Francisco, CA (US)

(21) Appl. No.: 14/783,776

(22) PCT Filed: Apr. 9, 2014
(86) PCT No.: PCT/US2014/033491

§ 371 Date: Oct. 9, 2015

Related U.S. Application Data
(60) Provisional application No. 61/810,643, filed on Apr. 10, 2013.

(51) Int. Cl. A61K 31/675; A61K 51/04

(52) U.S. Cl. CPC A61K 31/675; A61K 51/0453

(57) ABSTRACT

Cancer patients likely to respond to HAP treatment exhibit tumor tissues with high levels of hypoxia, which can be measured using PET imaging with [18F]-HX4.

### FIELD OF THE INVENTION

The present invention provides methods for predicting whether a cancer patient will respond favorably to TH-302 or another hypoxia activated prodrug (HAP) based anti-cancer therapy, and methods of treating cancer in such cancer patients, and so relates to the fields of biology, chemistry, and medicine.

### BACKGROUND OF THE INVENTION

Cancer is one of the major causes of human morbidity and mortality. Cancer treatment is challenging because it is difficult to kill cancer cells without damaging or killing normal cells. Damaging or killing normal cells during cancer treatment causes adverse side effects in patients and can limit the amount of anti-cancer drug administered to a cancer patient. It is also difficult to kill cancer cells in regions distant from the vasculature where anti-cancer drugs fail to penetrate.

Many cancer cells are more hypoxic relative to normal cells. Tumor hypoxia is associated with resistance to anti-cancer therapies, cancer relapse, and poor prognosis. Certain drugs in preclinical and clinical development target hypoxic cancer cells. These drugs, called HAPs, are administered in an inactive, or prodrug, form but are activated, and become toxic, in a hypoxic environment. PCT Pat. App. Pub. Nos. WO 07/002931 and WO 08/083101, each of which is incorporated herein by reference, describe HAPs such as those having a structure defined by Formula I, below:

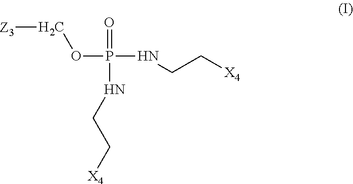

where Z₃ is selected from the group consisting of:

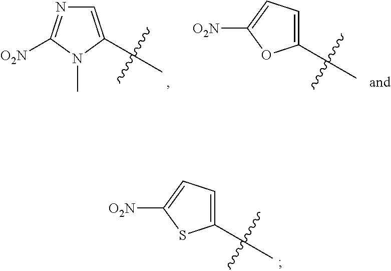

and X₄ is Cl or Br. The compounds known as TH-302 and TH-281 are particularly promising therapeutic candidates. TH-302, known by the chemical name (2-bromoethyl)({[(2-bromoethyl)amino][(2-nitro-3-methylimidazol-4-yl)methoxy]phosphoryl})amine, has the structure represented below:

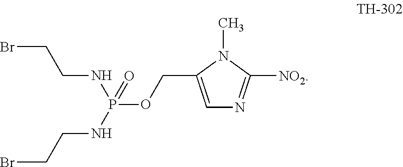 [SMILES: Cn1c(COP(=O)(NCCBr)NCCBr)cnc1[N+](=O)[O-]]

See Duan et al., 2008, “Potent and highly selective hypoxia-activated achiral phosphoramidate mustards as anti-cancer drugs,” J. Med. Chem. 51: 2412, incorporated herein by reference. TH-281 differs from TH-302 in that it has 2-chloroethyl groups instead of the 2-bromoethyl groups present in TH-302.

TH-302 is a hypoxia-activated prodrug composed of a 2-nitroimidazole oxygen concentration-sensitive trigger conjugated to a DNA cross-linking bromo-isophosphoramide mustard (Br-IPM) cytotoxic effector. TH-302 exhibits a broad efficacy in preclinical models and promising activity profile in multiple on-going clinical trials. See U.S. Pat. No. 8,003,625 and PCT Pat App. Pub. Nos. WO 2008/083101, WO 2010/048330, WO 2012/006032, WO 2012/009288, WO 2012/135757; WO 2012/142420, WO 2013/096684, WO 2013/096687, WO 2013/116385, and WO 2013/126539, each of which is incorporated herein by reference.

However, while nearly all tumors contain hypoxic regions, there is a wide variability among patients in how hypoxic a tumor of a given cancer type may be. For example, using median tumor pO₂ (mm Hg) as a measure of tumor hypoxia, one study of 33 soft tissue sarcoma patients showed that the median tumor pO₂ ranged from about 1 to about 70 mm Hg (see Nordsmark et al., 2001, Brit. J. Cancer 84(8): 1070-1075). Another study of 58 head and neck cancer patients showed the hypoxic fraction ranged from just above 90% to 1%. Thus, if greater tumor hypoxia correlates with a better response to HAP-mediated anti-cancer therapy, then this variability in tumor hypoxia will translate into a variable response to HAP anti-cancer therapy.

Positron emission tomography (PET) imaging employing the tracer [18F]-HX4 is suitable for imaging hypoxic tissue. [18F]-HX4 has the structure of formula:

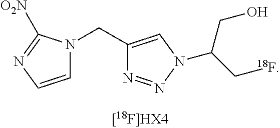

See U.S. Pat. Nos. 7,807,394 and 7,977,361, and Dubois et al. Proc. Natl. Acad. Sci. USA, 2011; 108:14620-14625, each of which is incorporated herein by reference. Methods to quantify the extent and magnitude of tumor hypoxia by PET imaging have been developed. See Mortensen et al. Radiother Oncol 2012; 105:14-20, which is incorporated herein by reference.

There remains a need for new methods to identify cancer patients likely to respond favorably to TH-302 or another HAP, both prior to the initiation of treatment and after TH-302 or another HAP administration has begun. The present invention meets this need.

### SUMMARY OF THE INVENTION

The present invention provides methods for identifying cancer patients likely to respond to TH-302 or another HAP based therapy. Generally, in these methods, [18F]-HX4 PET imaging or PET imaging employing another PET imaging agent is used to provide a measure of the hypoxic status of the cancer, i.e., how much hypoxic tumor or other cancerous tissues and cells are in the patient. In accordance with the methods of the invention, TH-302 or another HAP therapy is initiated, or, if already initiated, continued, if the hypoxic status is greater than a predetermined value. The predetermined value may be selected from values established from testing conducted on healthy subjects, or from testing conducted on other cancer patients, or by other means, but generally, the more hypoxic a patient's cancer, the more likely the patient will respond favorably to TH-302 or another HAP based anti-cancer therapy. The present invention also provides methods for treating cancer in a patient depending on the hypoxic status of the cancer in the patient.

### DETAILED DESCRIPTION OF THE INVENTION

### Definitions

The following definitions are provided to assist the reader. Unless otherwise defined, all terms of art, notations, and other scientific or medical terms or terminology used herein are intended to have the meanings commonly understood by those of skill in the chemical and medical arts. In some cases, terms with commonly understood meanings are defined herein for clarity and/or for ready reference, and the inclusion of such definitions herein should not be construed as representing a substantial difference over the definition of the term as generally understood in the art.

“A,” “an,” and, “the” include plural referents unless the context clearly dictates otherwise. Thus, for example, reference to a compound refers to one or more compounds or at least one compound. As such, the terms “a” (or “an”), “one or more”, and “at least one” are used interchangeably herein.

As used herein, the term “comprising” is intended to mean that the compositions and methods include the recited elements, but not excluding others. “Consisting essentially of” when used to define compositions and methods, shall mean excluding other elements of any essential significance to the composition or method. “Consisting of” shall mean excluding more than trace elements of other ingredients for claimed compositions and substantial method steps. Embodiments defined by each of these transition terms are within the scope of this invention. Accordingly, it is intended that the methods and compositions can include additional steps and components (comprising) or alternatively including steps and compositions of no significance (consisting essentially of) or alternatively, intending only the stated method steps or compositions (consisting of).

“Administering” or “administration of” a drug to a patient (and grammatical equivalents of this phrase) refers to direct administration, which may be administration to a patient by a medical professional or may be self-administration, and/or indirect administration, which may be the act of prescribing a drug. For example, a physician who instructs a patient to self-administer a drug and/or provides a patient with a prescription for a drug is administering the drug to the patient.

“Cancer” refers to leukemias, lymphomas, carcinomas, and other malignant tumors, including solid tumors, of potentially unlimited growth that can expand locally by invasion and systemically by metastasis. Examples of cancers include, but are not limited to, cancer of the adrenal gland, bone, brain, breast, bronchi, colon and/or rectum, gallbladder, head and neck, kidneys, larynx, liver, lung, neural tissue, pancreas, prostate, parathyroid, skin, stomach, and thyroid. Certain other examples of cancers include, acute and chronic lymphocytic and granulocytic tumors, adenocarcinoma, adenoma, basal cell carcinoma, cervical dysplasia and in situ carcinoma, Ewing's sarcoma, epidermoid carcinomas, giant cell tumor, glioblastoma multiforma, hairy-cell tumor, intestinal ganglioneuroma, hyperplastic corneal nerve tumor, islet cell carcinoma, Kaposi's sarcoma, leiomyoma, leukemias, lymphomas, malignant carcinoid, malignant melanomas, malignant hypercalcemia, marfanoid habitus tumor, medullary carcinoma, metastatic skin carcinoma, mucosal neuroma, myeloma, mycosis fungoides, neuroblastoma, osteo sarcoma, osteogenic and other sarcoma, ovarian tumor, pheochromocytoma, polycythermia vera, primary brain tumor, small-cell lung tumor, squamous cell carcinoma of both ulcerating and papillary type, hyperplasia, seminoma, soft tissue sarcoma, retinoblastoma, rhabdomyosarcoma, renal cell tumor, topical skin lesion, veticulum cell sarcoma, and Wilm's tumor.

“Hypoxia activated prodrug” or “HAP” refers to a prodrug wherein the prodrug is less active or inactive, relative to the corresponding drug, and comprises the drug and one or more bioreducible groups. HAPs include prodrugs that are activated by a variety of reducing agents and reducing enzymes, including without limitation single electron transferring enzymes (such as cytochrome P450 reductases) and two electron transferring (or hydride transferring) enzymes. In some embodiments, HAPs are 2-nitroimidazole triggered hypoxia-activated prodrugs. Examples of HAPs include, without limitation, TH-302, TH-281, PR104 and AQ4N. Methods of synthesizing TH-302 are described in PCT Pat. App. Pub. Nos. WO 07/002931 and WO 08/083101, incorporated herein by reference. Methods of synthesizing PR104 are described in US Pat. App. Pub. No. 2007/0032455, incorporated herein by reference. Other examples of HAPs are described, for example, in US Pat. App. Pub. Nos. 2005/0256191, 2007/0032455 and 2009/0136521 (each of which is incorporated herein by reference) and PCT Pat. App. Pub. Nos. WO 00/064864, WO 05/087075, and WO 07/002931 (each of which is incorporated herein by reference).

“Hypoxic status” refers to the level of hypoxia in cancer cells and/or tissues, such as in a patient's cancer cells and/or tissues. Such can be measured by PET imaging employing HX4 or another PET imaging agent.

“PET imaging” refers to imaging of tissue using positron emission tomography. As used herein, PET imaging includes imaging of tissue using “PET-CT”, which is a medical imaging technique that uses a device which combines in a single gantry system both a positron emission tomography (PET) scanner and an x-ray computed tomography (CT) scanner, so that images acquired from both devices can be taken sequentially, in the same session, and combined into a single superposed (co-registered) image.

“Patient” and “subject” are used interchangeably to refer to a mammal in need of treatment for cancer. Generally, the patient is a human. Generally, the patient is a human diagnosed with cancer. In certain embodiments a “patient” or “subject” may refer to a non-human mammal used in screening, characterizing, and evaluating drugs and therapies, such as, a non-human primate, a dog, cat, rabbit, pig, mouse or a rat.

“Prodrug” refers to a compound that, after administration, is metabolized or otherwise converted to a biologically active or more active compound (or drug) with respect to at least one property. A prodrug, relative to the drug, is modified chemically in a manner that renders it, relative to the drug, less active or inactive, but the chemical modification is such that the corresponding drug is generated by metabolic or other biological processes after the prodrug is administered. A prodrug may have, relative to the active drug, altered metabolic stability or transport characteristics, fewer side effects or lower toxicity, or improved flavor (for example, see the reference Nogrady, 1985, Medicinal Chemistry A Biochemical Approach, Oxford University Press, New York, pages 388-392, incorporated herein by reference). A prodrug may be synthesized using reactants other than the corresponding drug.

“Solid tumor” refers to solid tumors including, but not limited to, metastatic tumors in bone, brain, liver, lungs, lymph node, pancreas, prostate, skin and soft tissue (sarcoma).

“Therapeutically effective amount” of a drug refers to an amount of a drug that, when administered to a patient with cancer, will have the intended therapeutic effect, e.g., alleviation, amelioration, palliation or elimination of one or more manifestations of cancer in the patient. A therapeutic effect does not necessarily occur by administration of one dose, and may occur only after administration of a series of doses. Thus, a therapeutically effective amount may be administered in one or more administrations.

“Treating,” “treatment of,” or “therapy of” a condition or patient refers to taking steps to obtain beneficial or desired results, including clinical results. For purposes of this invention, beneficial or desired clinical results include, but are not limited to, alleviation or amelioration of one or more symptoms of cancer; diminishment of extent of disease; delay or slowing of disease progression; amelioration, palliation, or stabilization of the disease state; or other beneficial results. Treatment of cancer may, in some cases, result in partial response or stable disease.

### Descriptive Embodiments

Three types of biomarkers are generally recognized. Prognostic biomarkers correlate with disease outcome, independent of therapy, and include, for example, those used to establish “Triple Negative” breast cancer (ER−/PR−/HER−), which is generally believed to correlate with a poor prognosis. Response biomarkers measure the magnitude of the response to therapy and so can be viewed as analogous to RECIST response assessment. Response biomarkers include, for example, CA-19.9, where decreases in circulating CA-19.9 in pancreatic patients correlate with a positive response to therapy, and PSA, where, again, decreases in circulating PSA correlate with a positive response to therapy in prostate cancer patients. Predictive biomarkers are used before therapy to stratify or select patients for a therapy, e.g. HER2 profiling in breast cancer is used to determine suitability for trastuzumab therapy, and BRAF V600E profiling in melanoma is used to determine suitability for vemurafenib therapy.

Hypoxia is a prevalent feature of solid tumors, and hypoxia is a prognostic biomarker associated with treatment failure, poor prognosis, and increased metastasis. PET imaging with [18F]-HX4 (also referred to herein as “HX4”) or another PET imaging agent provides a measure of hypoxia. TH-302, a hypoxia-activated prodrug of the DNA cross-linker bromo isophosphoramide mustard, has demonstrated broad efficacy in preclinical models and is exhibiting promising activity in multiple ongoing clinical trials, both as a monotherapy and in combination therapy regimens. In accordance with the methods of the invention, PET imaging with [18F]-HX4 or another PET imaging agent is used to establish a hypoxic value for a patient's cancer, and if that value meets or exceeds a predetermined value, TH-302 or another HAP based anti-cancer therapy is initiated, or, if treatment was previously initiated, continued.

In one aspect, the present invention provides a method for determining whether a cancer patient should be administered TH-302 or another HAP, said method comprising (i) obtaining a measure of the hypoxic status of the cancer comprising using [18F]-HX4 PET imaging or PET imaging employing another PET imaging agent; and (ii) comparing the measure of hypoxic status obtained with a predetermined value, wherein if said measure is equal to or greater than said predetermined value, a determination to administer TH-302 or another HAP is made, and if not, then TH-302 or another HAP is not administered.

In one embodiment, said patient has not previously been treated with TH-302 or another HAP. In other embodiments, said patient has previously been treated with TH-302 or another HAP. In various embodiments, the PET imaging agent is [18F]-HX4.

In another aspect, the present invention provides a method for treating cancer in a patient, comprising:
obtaining a measure of the hypoxic status of the cancer using [18F]-HX4 PET imaging or PET imaging employing another PET imaging agent;
comparing the measure of hypoxic status obtained with a predetermined value; and
administering a therapeutically effective amount of TH-302 or another HAP to said patient if said measure is equal to or greater than said predetermined value, or administering a therapeutically effective amount of a cancer treatment other than a treatment comprising administration of a HAP if such measured level does not exceed said predetermined value.

In another aspect, the present method provides a method for treating cancer in a patient that has high hypoxic status in the cancer relative to the corresponding normal tissue, the method comprising administering a therapeutically effective amount of a HAP to the patient, wherein the hypoxic status is measured comprising using [18F]-HX4 PET imaging or PET imaging employing another PET imaging agent. In one embodiment, the HAP is TH-302. In various embodiments, the patient is treatment naïve.

In one embodiment, said patient has not previously been treated with TH-302 or another HAP. In another embodiment, said patient has previously been treated with TH-302 or another HAP. In various of these embodiments, the PET imaging agent is [18F]-HX4.

Certain non-limiting examples of PET imaging agents other than [18F]-HX4 that are suitable for use in the methods of the invention are shown below:

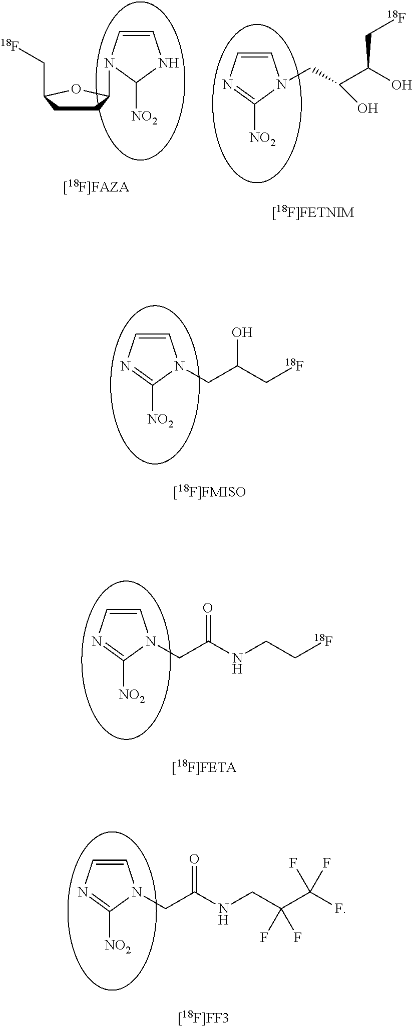

In certain embodiments, a “predetermined value” for hypoxic status, as used herein, is selected so that a patient with a hypoxic status higher than or equal to the predetermined value is likely to experience a more desirable clinical outcome than patients with hypoxic status lower than the predetermined value. One of skill in the art can determine such predetermined values by measuring hypoxic status in a patient population, e.g., following PET imaging, to provide a predetermined value. Optionally, a predetermined value for hypoxic status in one patient population can be compared to that from another to optimize the predetermined value to provide a higher predictive value. In various embodiments, a predetermined value refers to value(s) that best separate patients into a group with more desirable clinical outcomes and a group with less desirable clinical outcomes. Such predetermined value(s) can be mathematically or statistically determined with methods well known in the art in view of this disclosure. See, for example, Mortensen et al., supra (incorporated herein by reference).

Preparation and formulation of TH-302 or another HAP and methods of treating cancer employing TH-302 or another HAP are described for example in PCT Pat. App. Pub. Nos. WO 2007/002931, WO 2008/083101, WO 2010/048330, WO 2012/006032, WO 2012/009288, WO 2012/135757; WO 2012/142420, WO 2013/096684, WO 2013/096687, WO 2013/116385, and WO 2013/126539 (each of which is incorporated herein by reference), and can be adapted in accordance with the present invention by the skilled artisan.

As used herein, “a cancer treatment other than a treatment comprising administration of a HAP,” refers to treatments with one or more of non-HAP anti-cancer agents, radiation, and surgery. Suitable non-HAP anti-cancer agents useful for treating various cancers, and methods of their use, are well known to the skilled artisan, and described for example in a 2010 or more current edition of the Physician's Desk Reference, Medical Economics Company, Inc., Oradell, N.J.; Goodman and Gilman's The pharmacological basis of therapeutics., Eds. Hardman et al., McGraw-Hill. New York. (US) 2011, 12th Ed., and in publications of the U.S. Food and Drug Administration and the NCCN Guidelines (National Comprehensive Cancer Network). Such described and known methods can be appropriately modified by one of skill in the art, in view of this disclosure, to practice the treatment methods of the present invention.

In another aspect, the present invention provides a kit of parts comprising [18F]-HX4 or another PET imaging agent or a precursor thereof, and TH-302 or another HAP, and optionally an instruction for determining the hypoxic status of a cancer in a cancer patient and/or an instruction for administering TH-302 or another HAP to said patient. In one embodiment, the present invention provides a kit of parts comprising [18F]-HX4 (or a precursor) and TH-302, and optionally an instruction for determining the hypoxic status of a cancer in a cancer patient and/or an instruction for administering TH-302 to said patient.

The efficacy of the invention was demonstrated using animal models established with the H460 human non-small cell lung cancer (NSCLC) cell line and the 786-O human renal cell carcinoma (RCC) cell line. H460 is a high hypoxic fraction tumor model, while the 786-O RCC model is well-vascularized with very low hypoxia. Both [18F]-HX4 PET imaging of tumor-bearing animals and pimonidazole (pimonidazole is an agent used to detect hypoxic tissues and cells) immunostaining of tumor sections isolated from the same animals showed the same trend in the evaluation of hypoxia in the xenograft tumors, and which was related to TH-302 antitumor (anti-cancer) activity. When compiling the data from both H460 and 786-O tumors, there was significant correlation between [18F]-HX4 uptake and pimonidazole-positive hypoxic fraction (HF %, R2=0.55, p=0.006).

The utility of [18F]-HX4 to serve as a predictive biomarker for TH-302 anti-cancer efficacy was shown by the significant correlation observed between the TH-302 anti-cancer efficacy in a model and its hypoxic fraction as measured by [18F]-HX4. Employing the same treatment regimen of a dose of 50 mg/kg TH-302 administered on a schedule of QDx5 for 2 wk (daily for five days, then two days off, then daily for five days) with an intraperitoneal (i.p.) route of administration, TH-302 exhibits an anti-cancer efficacy, as measured by an inhibition of tumor growth kinetics when compared to vehicle treated animals, in the H460 model of TGD₅₀₀ of 18 days and TGI of 89% with a P value versus vehicle<0.001; and in the 786-O model of TGD₅₀₀ of 10 days, and TGI of 10% with a P value versus vehicle of >0.05. TGD₅₀₀ was determined as the increased time (days) for the treated tumor's size on average to reach 500 mm³ as compared with the vehicle group. TGI was defined as (1−ΔT/ΔC)×100, where ΔT/ΔC is the ratio of the change in mean tumor volume of the treated group (ΔT) and of the control group (ΔC).

The H460 xenograft tumors exhibit a poorly vascularized, highly hypoxic, mesenchymal phenotype. In the H460 xenograft model, three days (72 hrs) after TH-302 treatment (one dose at 150 mg/kg i.p.), tumor volume did not change significantly; however, the [18F]-HX4 uptake as measured by PET-computed tomography (PET-CT) was significantly reduced. When the animals were treated with TH-302 at a dose of 150 mg/kg, administered intraperitoneally (i.p.) in a Q4-5Dx3 (dosing once every four or five days for a total of three doses) regimen, tumors with higher initial [18F]-HX4 uptake grew slower, demonstrating better TH-302 response in more hypoxic tumors. Consistent with this result, the circulating plasma carbonic anhydrase 9 (CA-IX) level in these animals at this time point was significantly reduced as well (CA-IX is another biomarker of hypoxia; see PCT Pat. App. Pub. No. WO2013/116385, incorporated herein by reference); conversely, doxorubicin treatment (at a dose of 4 mg/kg administered intravenously (iv)) did not change circulating CA-IX levels significantly. Also, at this time point, 72 hours after TH-302 treatment, the hypoxic fraction (HF %) as semi-quantified by pimonidazole immunostaining was significantly reduced, which was consistent with the finding of reduced [18F]-HX4 uptake and reduced plasma circulating CA-IX level. Proliferating cells (as measured by Ki-67 immunostaining) were decreased as well; DNA damage (as measured by γH2AX immunostaining) and necrosis (as measured by hematoxylin and eosin, H&E, staining) were increased. There was regional co-localization of pimonidazole and CA-IX, but not co-localization of pimonidazole and two other putative endogenous hypoxia markers: Glut-1 or HIF-1α.

These preclinical results demonstrate that [18F]-HX4 PET imaging can guide TH-302 treatment decisions. PET imaging with [18F]-HX4 or another PET imaging agent can be used before the initiation of therapy to help guide the decision of whether TH-302 or another HAP should be employed for the patients' treatment. PET imaging with [18F]-HX4 or another PET imaging agent can also be use after the initiation of TH-302 or another HAP based therapy to help guide the decision of whether to continue to the use of TH-302 or another HAP based for the patients' treatment.

### EXAMPLE

The human H460 non-small cell lung cancer human tumor ectopic xenograft model was employed for this study. The H460 non-small cell lung cancer xenograft model was established by s.c. implantation of 1×10⁶ cells in the flank of nu/nu mice. When the tumor size was 100 mm³, a single dose of TH-302 (150 mg/kg, i.p.) or vehicle was administered. This dose of TH-302 yielded a TGI (tumor growth inhibition) of 58%, a TGD₁₀₀₀ (tumor growth delay to tumor size of 1000 mm³) of 10 days, and maximum body weight loss of 2%. The efficacy of TH-302 in individual animals varied in that the TGI varied from 6.3% to 105%.

Three biomarker platforms were employed in this study: positron emission tomography (PET) imaging employing the tracer [18F]-HX4; immunohistochemistry of isolated tumor tissue employing antibodies directed against the exogenous hypoxia biomarker pimonidazole and the endogenous hypoxia biomarker carbonic anhydrase IX (CA-IX); and ELISA-based quantification of circulating plasma CA-IX.

[18F]-HX4 uptake was assessed by PET/CT imaging, and the tumor to muscle ratio (T/M) was determined. Animals were scanned pre- and post-treatment, and the T/M ratio was significantly reduced 72 hours after TH-302 treatment (from 3.3±0.3 to 2.1±0.2, p<0.005). A slight increase in T/M was observed in the vehicle-treated animals (3.3±0.4 to 3.6±0.2, p>0.05). Blood was collected from the pre- and post-treated animals via retro-orbital venipuncture. Plasma CA-IX changed from baseline 165±13 to 104±9.3 pg/ml 72 hours after TH-302 dosing, a 37% reduction (p<0.05; n=10 per group). No change was observed in the vehicle-treated group or doxorubicin treated group (4 mg/kg, iv). For tissue biomarker analysis, tumors were harvested 72 hours after TH-302 treatment, and tumor hypoxia was assessed by semi-quantitative morphometric analysis of the PIMO immunostaining at that time. The hypoxic fraction (HF) was 13±1.7% in the vehicle group (n=5) and 5.8±0.5% in the TH-302 treatment group (n=6, p<0.05). Strong co-localization of CA-IX and PIMO expression was observed in consecutive sections, but Glut-1 or HIF-1α was not associated with these biomarkers. Proliferating cells, detected by Ki67, were significantly reduced after TH-302 treatment, while necrosis (as determined by hematoxylin and eosin staining) and DNA damage (by γH2AX) were significantly increased.

It should be understood that although the present invention has been specifically disclosed by certain aspects, embodiments, and optional features, modification, improvement and variation of such aspects, embodiments, and optional features can be resorted to by those skilled in the art, and that such modifications, improvements and variations are considered to be within the scope of this disclosure.

The inventions have been described broadly and generically herein. Each of the narrower species and subgeneric groupings falling within the generic disclosure also form part of the invention. In addition, where features or aspects of the invention are described in terms of Markush groups, those skilled in the art will recognize that the invention is also thereby described in terms of any individual member or subgroup of members of the Markush group.

1. A method for determining whether a cancer patient should be administered TH-302, said method comprising (i) obtaining a measure of the hypoxic status of the cancer using [18F]-HX4 PET imaging; and (ii) comparing the measure of hypoxic status obtained with a predetermined value, wherein if said measure is equal to or greater than said predetermined value, a determination to administer TH-302 is made, and if not, then TH-302 is not administered.

2. The method of claim 1, wherein said patient has not previously been treated with TH-302.

3. The method of claim 1, wherein said patient has previously been treated with TH-302.

4. A method for treating cancer in a patient, comprising:
obtaining a measure of the hypoxic status of the cancer using [18F]-HX4 PET imaging;
comparing the measure of hypoxic status obtained with a predetermined value; and
administering a therapeutically effective amount of TH-302 to said patient if said measure is equal to or greater than said predetermined value, or administering a therapeutically effective amount of a cancer treatment other than a treatment comprising administration of TH-302 if such measured level does not exceed said predetermined value.

5. The method of claim 4, wherein said patient has not previously been treated with TH-302.

6. The method of claim 4, wherein said patient has previously been treated with TH-302.

7. A kit of parts comprising [18F]-HX4 and TH-302, and optionally an instruction for determining the hypoxic status of a cancer in a cancer patient and/or an instruction for administering TH-302 to said patient.

8. (canceled)
